(19) United States
(12) Patent Application Publication
(10) Pub. No.: US 2013/0116791 A1
(43) Pub. Date: May 9, 2013

(54) Expandable intervertebral spacer implant

(71) Applicant: BOO HOLDINGS, LLC, Palm Beach Gardens, FL (US)

(72) Inventor: Charles S. Theofilos, Palm Beach Gardens, FL (US)

(73) Assignee: BOO HOLDINGS, LLC, Palm Beach Gardens, FL (US)

(21) Appl. No.: 13/667,553

(22) Filed: Nov. 2, 2012

Related U.S. Application Data
(60) Provisional application No. 61/555,761, filed on Nov. 4, 2011.

(51) Int. Cl. A61F 2/44

(52) U.S. Cl. CPC A61F 2/442
USPC 623/17.16

(57) ABSTRACT

Expandable intervertebral spacer implants are illustrated for insertion into an intervertebral space between a superior vertebral body and an inferior vertebral body. The implant is formed as a first and second member having a variable distance therebetween, each member having a first end and a second end with a length therebetween, a proximal side and a distal side with a width therebetween, and a pair of internally guided legs which are rotatable about one or more common axes and are positioned to successively interlock with a plurality of detents formed within each said first and second members to facilitate maintenance of a specific distance therebetween.

### RELATED APPLICATION

In accordance with 37 C.F.R. 1.76, a claim of priority is included in an Application Data Sheet filed concurrently herewith. Accordingly, the present invention claims priority under 35 U.S.C. §119(e), 120, 121, and/or 365(c) to U.S. Provisional Patent Application No. 61/555,761, entitled “Expandable intervertebral spacer implant”, filed on Nov. 4, 2011. The contents of which the above referenced application is incorporated herein by reference in its entirety.

### FIELD OF THE INVENTION

This invention generally relates to expandable spinal cages, and particularly to expandable intervertebral spacer implants for insertion into an intervertebral space between a superior vertebral body and an inferior vertebral body. The implant is formed as a first and second member, each member having a first end and a second end with a length therebetween, a proximal side and a distal side with a width therebetween, and a pair of internally guided legs which are rotatable about a common axis or axes, and are positioned to successively interlock with a plurality of detents formed within each said first and second members to facilitate an expandable height therebetween.

### BACKGROUND OF THE INVENTION

Expandable cages are utilized by a spine surgeon attempting to restore stability in patients undergoing corpectomies. They are useful in cases ranging from destructive osteomyelitis, degenerative spine disease with myelopathy, and primary or metastatic tumors of the spinal column.

Correct positioning of traditional non-expandable cages can be challenging. Some distraction forces and shaping of the endplates may be necessary to insert a rigid implant. The positioning of the implant can often be inadequate, the endplates can weaken with consequent subsidence, and restoring sagittal alignment can be insufficient.

One of the main advantages of expandable cages for vertebral body replacement is easy, non-distracted insertion of the cage. The height can be adjusted to the corpectomy or disc space defect in situ, and correction of deformity and restoring height can be achieved. Expandable cages have excellent axial load-bearing capacity and they have the added benefit of allowing for morselized autograft/allogenic bone to be packed into the cage, thus enhancing the rate of fusion. Their advantages over simple cages are that by expanding they can precisely occupy the space created and that by exerting a longitudinal force they manage to primarily correct a deformity. Since they allow for less retraction during the operation, they are easier to maneuver. Simultaneously, these cages provide the advantage of being height adjustable, thus facilitating their insertion without cage cutting or problematic impaction of the spinal cord. Furthermore, the ability of expandable cages to reduce postoperative pain has been well documented in the literature, and is an important asset, especially in patients where quality of life is of major importance. However, for all of their benefits, one of the pitfalls of expandable cages is over-distraction.

The mechanical properties of the instantly disclosed implant allow for smooth and easy distraction, without requiring the surgeon to maneuver against too much resistance, thereby reducing the incidence of traction injury of the segmental nerves.

### SUMMARY OF THE INVENTION

This invention is directed towards expandable spinal cages, and particularly to expandable intervertebral spacer implants for insertion into an intervertebral space between a superior vertebral body and an inferior vertebral body. The implant is formed as a first and second member, each member having a first end and a second end with a length therebetween, a proximal side and a distal side with a width therebetween, and a pair of internally guided legs which are rotatable about a common axis or axes, and are positioned to successively interlock with a plurality of detents formed within each said first and second members to facilitate an expandable height therebetween.

Accordingly, it is a primary objective of the instant invention to provide an expandable intervertebral spacer implant for insertion into an intervertebral space between a superior vertebral body and an inferior vertebral body.

It is a further objective of the instant invention to provide said spacer implant as a first and second member, each member having a first end and a second end with a length therebetween, a proximal side and a distal side with a width therebetween, and a pair of internally guided legs which are rotatable about a common axis or axes, and are positioned to successively interlock with a plurality of detents formed within each said first and second members to facilitate an expandable height therebetween.

It is yet another objective of the instant invention to provide a system for insertion and distraction of said spacer implant.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A is an exploded view of the expandable intervertebral spacer implant in accordance with the present invention;

FIG. 1B is a side view showing the expandable intervertebral spacer implant in its fully collapsed orientation;

FIG. 1C is a side view showing the expandable intervertebral spacer implant in its partially collapsed orientation;

FIG. 1D is a side view showing the expandable intervertebral spacer implant in a distracted orientation;

FIG. 1E is a side view of an alternative leg configuration having a plurality of axes;

FIG. 2A is an exploded side view of a variable sized expander tool and a collapsed expandable intervertebral spacer implant;

FIG. 2B is a side view of a variable size expander tool engaged with an expandable intervertebral spacer implant in a partially distracted orientation;

FIG. 3A is a rear view of FIG. 1B;

FIG. 3B is a rear view of FIG. 1C;

FIG. 3C is a rear view of FIG. 1D.

### DETAILED DESCRIPTION OF THE INVENTION

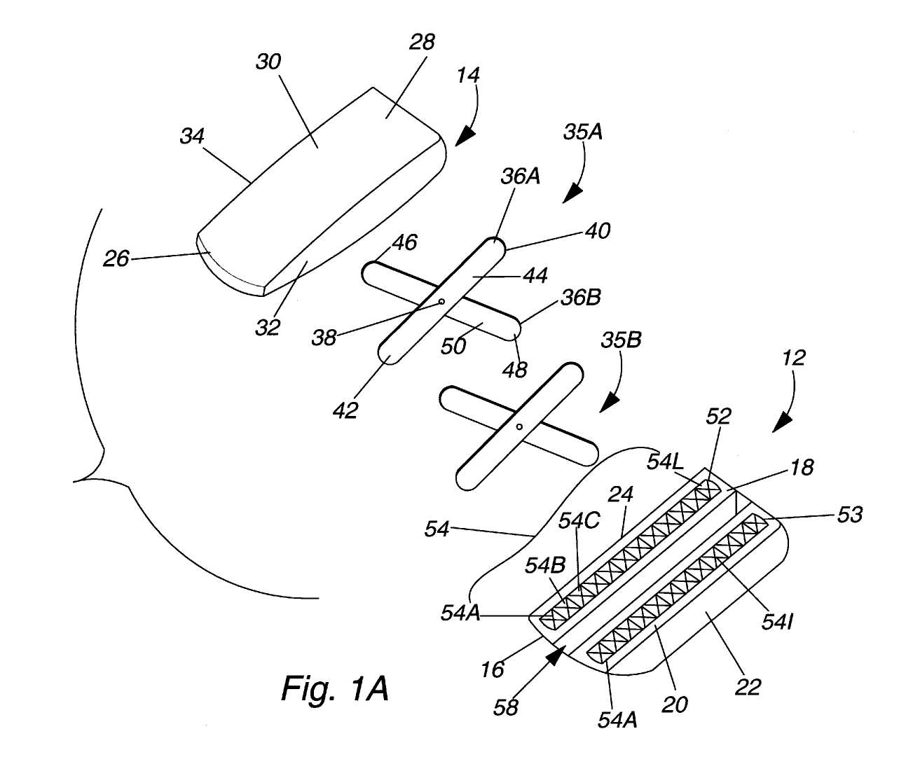

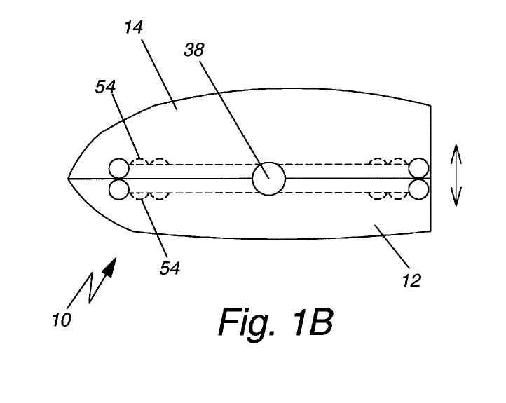

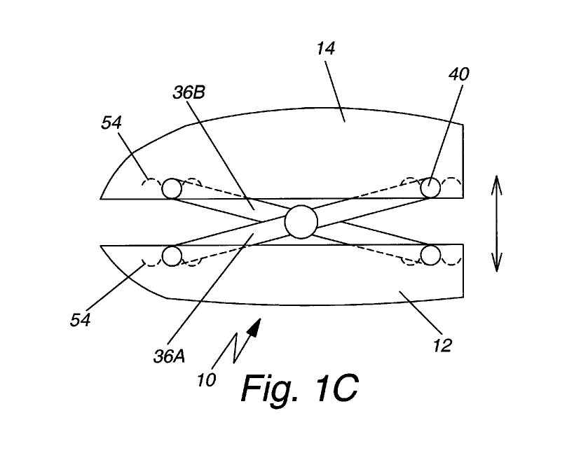

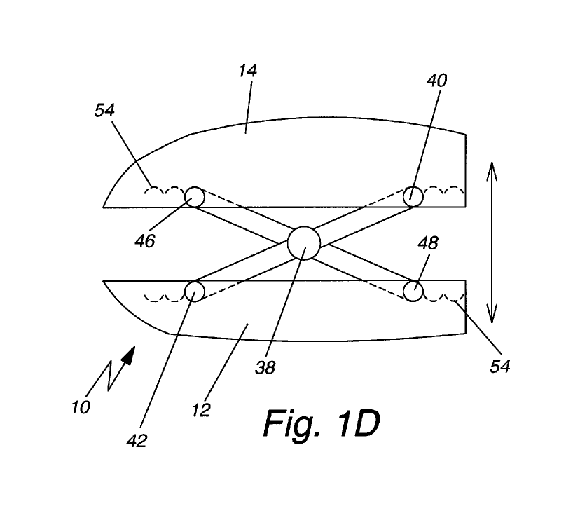

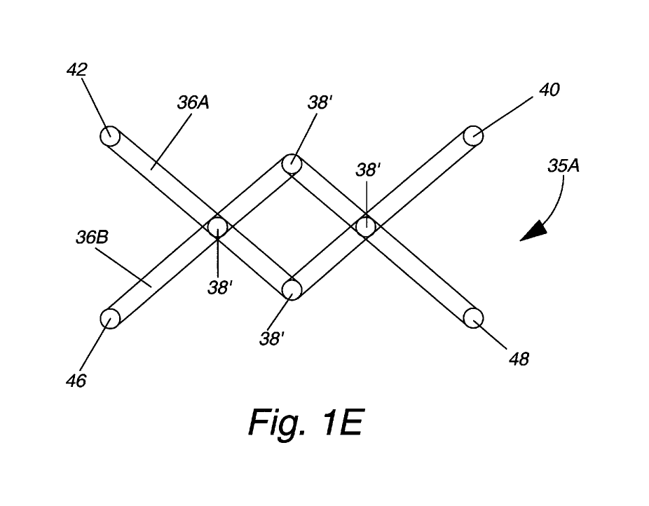

Referring to FIG. 1A, an expandable spinal cage implant, referred to generally as 10, is illustrated. The expandable spinal cage implant 10 contains a first member 12 and a second member 14. Both the first member 12, the second member 14, and all other components may be made of any material known to one of skill and which is useful for its intended purpose, i.e. insertion into an intervertebral space. In a preferred embodiment, the expandable spinal cage implant 10 is made of biocompatible materials. Additionally, the first member 12 and the second member 14 can sized and shaped according to the its intended use and/or desired implant location.

The first member 12 contains a first end 16, a second end 18, and a first member main body 20 defined by a length there between. The first member 12 further contains a proximal side 22, a distal side 24, and a width there between. The second member 14 contains a first end 26, a second end 28, and a second member main body 30 defined by a length there between. The second member 14 further contains a proximal side 32, a distal side 34, and a width there between. The first member 12 and the second member 14 preferably are of the same length and arranged in a parallel manner relative to each other when properly aligned, i.e. when the second member 12 is positioned above the first member 14. One or both members 12 and 14 may contain a tapered surface for ease of inserting the expandable intervertebral spacer implant 10 into the intervertebral space.

To create expansion capability, the expandable spinal cage implant 10 contains a plurality of expanders, referred generally as 35A and 35B. The expanders 35A and 35B preferably include a plurality of expanding members adapted to engage and move the first and second implant members 12, 14 apart. As an illustrative example, the expanders 35A and 35B are shown with a plurality of expanding members illustrated as pair of legs, a first leg 36A and a second leg 36B, pivotally connected through an axis point 38, illustrated as a pin. The first leg 36A contains a first end 40, a second end 42, and a first leg main body 44 therebetween. The second leg 36B contains a first end 46, a second end 48, and a second leg main body 50 therebetween. The second expander 35B is configured in the same manner as expander 35A. Each of the expanders 35A and 35B are couplable to the first member 12 and to the second member 14 to provide for expansion capability.

To accomplish such feature, the first member 12 contains a pair of internal tracks 52 and 53, preferably running substantially the distance between the first end 16 and the second end 18. However, the length of the tracks 52 and 53 may be varied depending on the use. While the internal tracks are illustrated as continuous tracks spanning the distance from end to end, the tracks may be made of several discontinuous tracks. In this embodiment, the expandable spinal cage implant 10 would utilize a plurality of expanders positioned within each discontinuous track.

While not illustrated, the second member 14 contains corresponding internal tracks having the same characteristics as described for tracks 52 and 53. Preferably the tracks associated with the first member 12 and the second member 14 are arranged in a parallel manner and aligned in a co-linear manner when both implant members are aligned together. To provide for expansion such that the first/second members moves relative to the other member, each ends 40, 42 of leg 36A and ends 46 and 48 of leg 36B engage the internal tracks of both the first member 12 and the second member 14. For example, the first end 40 is coupled to an internal track within the second member 14 (not illustrated) and the second end 42 is coupled to the internal track 52. Additionally, the second leg 36B connects to the first and second members 12 and 14 through coupling of the first end 46 to an internal track within the second member 14, and coupling the second end 48 to the internal track 52. The second expander 35B would couple in the same manner as described above using track 52 for the first member 12 and a corresponding track within the second member 14 but would utilize track 53 and a second track positioned within the second member 14. Each of the ends of the legs 36A or 36B, as well as the ends of any the legs associated with expanding member 35B, are positioned to successively interlock with a plurality of detents, referred to generally as 54, and individually as 54A, 54B, 54C, to 541 formed within each of the first member 12 and second member 14, whereby serial engagement with the detents 54 facilitate maintaining a desired expandable height or distance therebetween. Each detent 54 may include a cavity sized and shaped to engage each of the ends of the legs associated with the expanding members 35A and 35B.

Now referring to FIGS. 1B, 1C and 1D, a side view of the expandable spinal cage of the instant invention is shown. The expandable intervertebral spacer implant 10 is illustrated in a first fully collapsed position, see FIG. 1B, a second partially collapsed position, see FIG. 1C, and a third distracted position, see FIG. 10. As seen in FIGS. 1B-1D, each of the pairs of legs 36A and 36B are initially in their closed position, to allow for ease of insertion into an intervertebral space between a superior vertebral body and an inferior vertebral body. Each of the pairs of internally guided legs 36A and 36B are configured to be rotatable about either the single common axis 38, or about a plurality of axes 38′, see FIG. 1E. Each of the ends of the legs is also positioned to successively interlock with a plurality of detents 54 formed within each of the first and second members, whereby serial engagement with said detents 20 facilitate maintaining a desired expandable height or distance therebetween. As an illustrative example, the ends may be adapted to include rounded edges, ball like features, or may include a pin. Each end is preferably sized and shaped to be couplable with the one or more detent 54 cavities so as to secure the legs 36A and 36B therein. As the first member 12 and the second member 14 are successively distracted as shown in FIGS. 1C and 1D, the legs 36A and 36B remain slidably engaged within the internal tracks. This facilitates their positioning relative to the respective detents 54 dependent upon the desired degree of distraction.

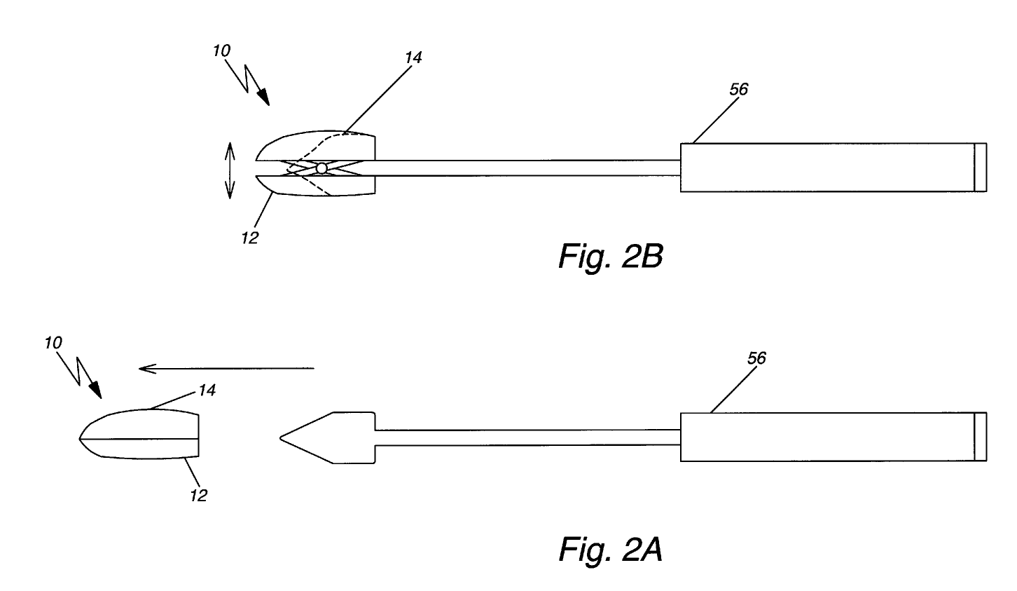

With reference to FIG. 2A, an exploded side view of a variable sized expander 56 is illustrated as it would be aligned for engagement with the collapsed expandable intervertebral spacer implant 10. FIG. 2B shows a side view of the variable size expander 56 engaged with the expandable intervertebral spacer implant 10, which is illustrated in a partially distracted orientation.

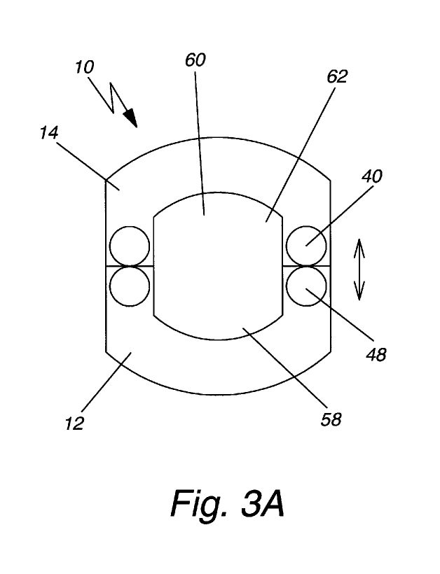

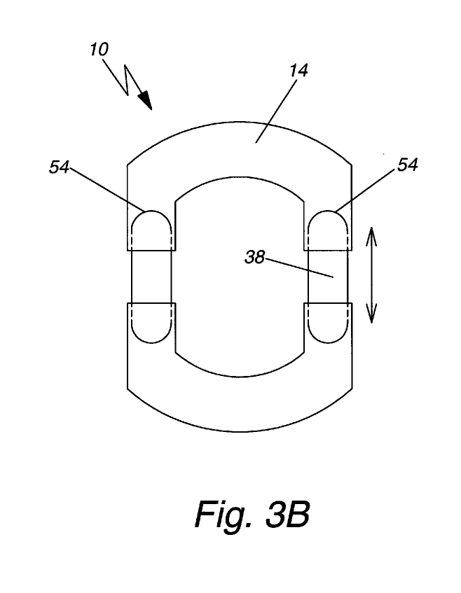

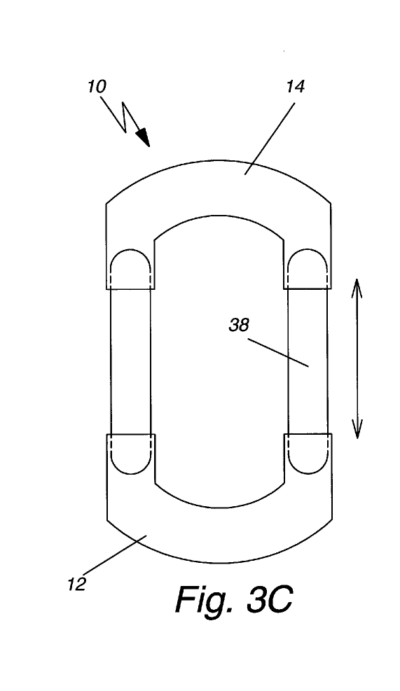

FIGS. 3A, 3B and 3C are rear facing views of the expandable intervertebral spacer implant as illustrated in FIGS. 1B, 1C and 1D, respectively. As illustrated, each of the first and second members 12, 14 may contain a channel 58 and 60, which when in the non-expanded state creates an opening or void space 62 within the expandable intervertebral spacer implant 10. Each of the channels 58 and 60 can be sized and shaped to receive one or more expanders 56, thereby providing for controlled expansion of the first member, the second member, or combinations thereof. The opening or void space 62 may also allow bone growth from adjacent vertebra there through and/or insertion of bone growth material within the expandable intervertebral spacer implant 10. Alternatively, or in addition to, the firs member 12 and the second member 14 may be adapted to be packable with bone growth material.

In a particular illustrative embodiment of the instant invention, a kit is provided containing an implant 10, along with a plurality of expanders 56, wherein successive serial engagement of the detents is enabled by proper selection of the appropriate expander 56. The kit may also include a plurality of expandable spinal cage implant 10 in accordance with the present invention in variable sizes and/or shapes.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An expandable intervertebral spacer implant adapted for insertion into an intervertebral space between a superior vertebral body and an inferior vertebral body comprising:
a first implant member having a first end, a second end, and a first implant member main body defined by a distance therebetween,
a second implant member having a first end, a second end, and a second implant member main body defined by a distance therebetween, whereby said first implant member and said second implant member being aligned in a substantially parallel manner in a non-expanded position; and
at least two expanders, each said expander having at least two expanding members constructed and arranged to be rotatable about a common axis, and further adapted to move said first implant member, said second implant member, or combinations thereof.

2. The expandable intervertebral spacer implant adapted for insertion into an intervertebral space between a superior vertebral body and an inferior vertebral body according to claim 1 wherein said two expanding members include a pair of internally guided legs constructed and arranged to be rotatable about said common axis, each said leg having ends adapted for concurrent engagement with a portion of said first implant member and said second implant member.

3. The expandable intervertebral spacer implant adapted for insertion into an intervertebral space between a superior vertebral body and an inferior vertebral body according to claim 1 wherein each said first member and each said second member contain a plurality of channels sized and shaped to receive at least one expander.

4. The expandable intervertebral spacer implant adapted for insertion into an intervertebral space between a superior vertebral body and an inferior vertebral body according to claim 1 wherein each said first member and each said second member contain one or more detents formed therein adapted for maintaining a distance between said first and second implant members.

5. The expandable intervertebral spacer implant adapted for insertion into an intervertebral space between a superior vertebral body and an inferior vertebral body according to claim 1 wherein each said expander comprises of at least two expanding members constructed and arranged to be rotatable about a plurality of axes.

6. The expandable intervertebral spacer implant adapted for insertion into an intervertebral space between a superior vertebral body and an inferior vertebral body according to claim 1 wherein said channels formed within said first member are substantially aligned with said channels formed within said second member.

7. The expandable intervertebral spacer implant adapted for insertion into an intervertebral space between a superior vertebral body and an inferior vertebral body according to claim 1 wherein said first implant member and said second implant member have tapered surfaces.

8. The expandable intervertebral spacer implant adapted for insertion into an intervertebral space between a superior vertebral body and an inferior vertebral body according to claim 1 wherein said first member and said second member are adapted to be packable with bone growth material.

9. An expandable intervertebral spacer implant kit comprising:
an expandable intervertebral spacer implant adapted for insertion into an intervertebral space between a superior vertebral body and an inferior vertebral body, said spacer comprising a first implant member having a first end, a second end, and a first implant member main body defined by a distance therebetween; a second implant member having a first end, a second end, and a second implant member main body defined by a distance therebetween, whereby said first implant member and said second implant member being aligned in a substantially parallel manner in a non-expanded position; and at least two expanders, each said expander having at least two expanding members constructed and arranged to be rotatable about a common axis, and further adapted to move said first implant member, said second implant member, or combinations thereof, and
a plurality of variably sized expander tools constructed and arranged for insertion within said expandable intervertebral spacer implant to enable successive degrees of distraction.

10. The expandable intervertebral spacer implant kit according to claim 9 further including a plurality of variably sized expandable intervertebral spacer implants.

11. The expandable intervertebral spacer implant kit according to claim 9 wherein said two expanding members include a pair of internally guided legs constructed and arranged to be rotatable about said common axis, each said leg having ends adapted for concurrent engagement with a portion of said first implant member and said second implant member.

12. The expandable intervertebral spacer implant kit according to claim 9 wherein each said first member and each said second member contain a plurality of channels sized and shaped to receive at least one expander.

13. The expandable intervertebral spacer implant kit according to claim 9 wherein each said first member and each said second member contain one or more detents formed therein adapted for maintaining a distance between said first and second implant members.

14. The expandable intervertebral spacer implant kit according to claim 9 wherein each said expander comprises of at least two expanding members constructed and arranged to be rotatable about a plurality of axes.

15. The expandable intervertebral spacer implant kit according to claim 9 wherein said channels formed in said first member are substantially aligned with said channels in said second member.
